(19) United States
(12) Patent Application Publication
(10) Pub. No.: US 2025/0041261 A1
(43) Pub. Date: Feb. 6, 2025

(54) METHODS AND COMPOSITIONS FOR TREATING MELANOMA

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); UNIVERSITE COTE D'AZUR, Nice (FR)

(72) Inventors: Corine BERTOLOTTO, Nice Cedex 3 (FR); Robert BALLOTTI, Nice Cedex 3 (FR); Raphaël RODRIGUEZ, Paris Cedex 05 (FR); Teresa Tatiana CANEQUE COBO, Paris Cedex 05 (FR)

(21) Appl. No.: 18/718,519

(22) PCT Filed: Dec. 20, 2022
(86) PCT No.: PCT/EP2022/087023

§ 371 Date: Jun. 11, 2024

(30) Foreign Application Priority Data
Dec. 21, 2021 (EP) 21306878.6

(51) Int. Cl. A61K 31/35; A61K 45/06; A61P 35/00

(52) U.S. Cl. CPC A61K 31/35; A61K 45/06; A61P 35/00

(57) ABSTRACT

Inventors have shown that salinomycin and its derivatives such as AM23, through lysosomal iron accumulation, reduced proliferation/survival of uveal melanoma cells harboring different genetic background. Accordingly, salinomycin and its derivates ironomycin represent valuable therapeutic strategies to treat melanoma and uveal melanoma. The present invention relates to a method for treating melanoma in a subject in need thereof comprising a step of administering said subject with a therapeutically effective amount of an iron metabolism disruptor.

### FIELD OF THE INVENTION

The invention is in the field of oncology, more particularly in the field of melanoma.

### BACKGROUND OF THE INVENTION

Both cutaneous and uveal melanoma are aggressive and deadly neoplasms, which develop from melanocytes located in the skin and uvea respectively.

Major breakthroughs were realized in the treatment of metastatic cutaneous melanoma this last decade with targeted therapies (BRAF and MEK inhibitors) and immunotherapies (anti-CTLA4, anti-PD1). However, despite the undeniable progress made by these new treatments, between 50 and 60% of patients are resistant or develop resistance, highlighting the need to find new therapeutic approaches to overcome or prevent this resistance.

Uveal melanoma and cutaneous melanoma show significant differences in the etiologic factors and mutational status. Metastatic uveal melanomas are highly refractory to existing therapies, including not surprisingly, those that improve the overall survival of patient with cutaneous melanoma. Recently bispecific fusion protein tebentafusp (IMC-gp100), has been shown to improve the overall survival of HLA-A*02:01 positive mUM patients. However, despite this breakthrough, most uveal melanoma patients will die within 6 months after diagnosis of metastases, highlighting an urgent need for identification of efficient therapeutic strategies to improve their survival.

### SUMMARY OF THE INVENTION

The present invention relates to a method for treating melanoma in a subject in need thereof comprising a step of administering said subject with a therapeutically effective amount of an iron metabolism disruptor. In particular, the invention is defined by the claims.

### DETAILED DESCRIPTION OF THE INVENTION

Inventors have treated a panel of uveal melanoma cells, i.e the human metastatic OMM1.3 (BAP1+) and MM28 (BAP1+) cells, and the human primary MP46 (BAP1−) and MP65 (BAP1−) cells, with Salinomycin (Sal) or its derivate AM23 to determine if these drugs can represent potential therapeutic options. Data show that both Sal and AM23 reduced proliferation/survival of uveal melanoma cell in 2D-cultures (FIG. 1). Additionally, as observed by almost complete disappearance of melanospheres at 100 nM, AM23 was as well highly effective in reducing uveal melanoma cell survival in 3D-cultures that much more closely resemble the cells growing within living tumors (FIG. 2).

The results suggest that Sal and AM23, through lysosomal iron accumulation, reduced proliferation/survival of uveal melanoma cells harboring different genetic background. In conclusion, salinomycin and its derivates ironomycin represent valuable therapeutic strategies to treat melanoma and uveal melanoma.

### Method for Treating Melanoma

Accordingly, in a first aspect, the present invention relates to a method for treating melanoma in a subject in need thereof comprising a step of administering said subject with a therapeutically effective amount of an iron metabolism disruptor.

More particularly, the invention relates to an iron metabolism disruptor for use in the treatment of melanoma in a subject in need thereof.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “subject” denotes a mammal, such as a rodent, a feline, a canine, and a primate. Particularly, the subject according to the invention is a human.

More particularly, the subject according to the invention has or is susceptible to have melanoma.

In particular embodiment, the subject has or is susceptible to have cutaneous melanoma.

In a particular embodiment, the subject has or is susceptible to have metastatic melanoma.

In a particular embodiment, the subject has or is susceptible to have uveal melanoma.

In a particular embodiment, the subject has or is susceptible to have uveal melanoma resistant.

As used herein, the term “subject” encompasses “patient”.

As used herein, the term “melanoma” also known as malignant melanoma, refers to a type of cancer that develops from the pigment-containing cells, called melanocytes. There are three general categories of melanoma: 1) cutaneous melanoma which corresponds to melanoma of the skin; it is the most common type of melanoma; 2) mucosal melanoma which can occur in any mucous membrane of the body, including the nasal passages, the throat, the vagina, the anus, or in the mouth; and 3) ocular melanoma also known as uveal melanoma or choroidal melanoma, is a rare form of melanoma that occurs in the eye.

In a particular embodiment, the melanoma is cutaneous melanoma.

In a particular embodiment, the melanoma is uveal melanoma. More particularly, the invention relates to an iron metabolism disruptor for use in the treatment of uveal melanoma in a subject in need thereof.

As used herein, the term “uveal melanoma” refers to a disease in which malignant (cancer) cells form in the tissues of the eye. It is an aggressive and deadly neoplasm, which develops from melanocytes in the choroid. At diagnosis, only 1-3% of the patients have detectable metastases.

As used herein the terms “administering” or “administration” refer to the act of injecting or otherwise physically delivering a substance as it exists outside the body (e.g., iron metabolism disruptor) into the subject, such as by topical, intravitreal, mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In a particular embodiment, the administration is a intravitreal administration. In another particular embodiment, the administration is a topical administration.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient (iron metabolism disruptor) for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

As used herein the term “iron metabolism disruptor” also called as inhibitor of iron metabolism refers to a natural or synthetic compound that has a biological effect on iron metabolism homeostasis. More particularly, such compound has an effect on iron chelation or cellular iron uptake. Typically, such deregulation triggers ferroptosis, a type of cell death based on accumulation of iron into lysosomes and subsequent lysosomal membrane permeabilization. More particularly, the drugs through lysosomal iron accumulation, reduces proliferation/survival of melanoma cells.

In a particular embodiment, the iron metabolism disruptor is a peptide, peptidomimetic, small organic molecule, antibody, aptamers, siRNA or antisense oligonucleotide. The term “peptidomimetic” refers to a small protein-like chain designed to mimic a peptide. In a particular embodiment, the iron metabolism disruptor is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, iron metabolism disruptor is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In a particular embodiment, the iron metabolism disruptor is salinomycin and its derivatives. Typically, salinomycin has the chemical formula C₄₂H₇₀O₁₁, IUPAC name (2R)-2-[(5S,6R)-6-[(1S,2S,3S,5R)-5-[(2S,5R,7S,9S,10S, 12R, 15R)-2-[(2R,5R,6S)-5-ethyl-5-hydroxy-6-methyl-2-tetrahydropyranyl]-15-hydroxy-2,10,12-trimethyl-1,6,8-trioxadispiro[4.1.57.35]pentadec-13-en-9-yl]-2-hydroxy-1,3-dimethyl-4-oxoheptyl]-5-methyl-2-tetrahydropyranyl]butanoic acid. Salinomycin has the following CAS number 53003-10-4 and structure in the art:

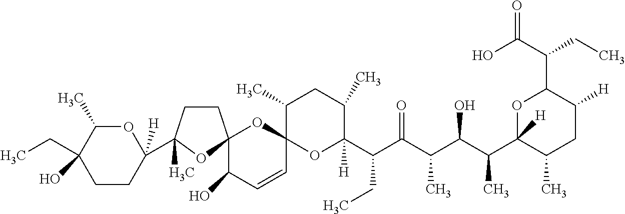

More particularly, salinomycin and its derivatives are described in WO2016/038223.

In another embodiment, the iron metabolism disruptor is ironomycin which is a derivative of salinomycin. Ironomycin also called as AM5 or AM23 is described in Trang Thi Mai et al 2017 (Nat Chem. 2017 October; 9 (10): 1025-1033. doi: 10.1038/nchem.2778.).

Typically, Ironomycin has the chemical formula C₄₅H₇₃NO₁₀. IUPAC name (2R)-2-[(2R,5S,6R)-6-{(2S,3S,4S,6R)-6-[(2S,5S,7R,9S,10S,12R,15R)-2-[(2R,5R,6S)-5-Ethyl-5-hydroxy-6-methyltetrahydro-2H-pyran-2-yl]-2, 10,12-trimethyl-15-(2-propyn-1-ylamino)-1,6,8-trioxadispiro[4.1.5.3]pentadec-13-en-9-yl]-3-hydroxy-4-methyl-5-oxo-2-octanyl}-5-methyltetrahydro-2H-pyran-2-yl]butanoic acid in the art. Ironomycin has the following structure in the art:

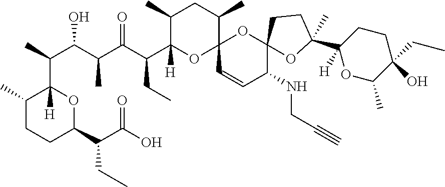

In a particular embodiment, the invention relates to an iron metabolism disruptor for use in the treatment of melanoma in a subject in need thereof.

In a particular embodiment, the invention relates to salinomycin and its derivatives as described above for use in the treatment of melanoma in a subject in need thereof.

In a particular embodiment, the invention relates to salinomycin and its derivatives as described above for use in the treatment of uveal melanoma in a subject in need thereof.

In some embodiments, the iron metabolism disruptor is a short hairpin RNA (shRNA), a small interfering RNA (siRNA) or an antisense oligonucleotide which inhibits the expression of metabolites involved in iron metabolism.

In a particular embodiment, the iron metabolism disruptor is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene.

In a particular embodiment, the iron metabolism disruptor is an anti-sense oligonucleotides (ASO). Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the iron metabolism disruptor is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339:823-826), bacteria (Fabre et al., 2014, PLOS Negl. Trop. Dis., Vol. 8: e2671.), zebrafish (Hwang et al., 2013, PLOS One, Vol. 8: e68708.), C. elegans (Hai et al., 2014 Cell Res. doi: 10.1038/cr.2014.11.), bacteria (Fabre et al., 2014, PLOS Negl. Trop. Dis., Vol. 8: e2671.), plants (Mali et al., 2013, Science, Vol. 339:823-826), Xenopus tropicalis (Guo et al., 2014, Development, Vol. 141:707-714.), yeast (DiCarlo et al., 2013, Nucleic Acids Res., Vol. 41:4336-4343.), Drosophila (Gratz et al., 2014 Genetics, doi: 10.1534/genetics.113.160713), monkeys (Niu et al., 2014, Cell, Vol. 156:836-843.), rabbits (Yang et al., 2014, J. Mol. Cell Biol., Vol. 6:97-99.), pigs (Hai et al., 2014, Cell Res. doi: 10.1038/cr.2014.11.), rats (Ma et al., 2014, Cell Res., Vol. 24:122-125.) and mice (Mashiko et al., 2014, Dev. Growth Differ. Vol. 56:122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In some embodiments, the iron metabolism disruptor is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388.

In a particular embodiment, the iron metabolism disruptor is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the iron metabolism disruptor is an intrabody. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

### Method for Treating Resistant Melanoma

In a second aspect, the invention relates to a method for treating resistant melanoma in a subject in need thereof comprising a step of administering said subject with a therapeutically effective amount of an iron metabolism disruptor.

As used herein, the term “resistant melanoma” refers to melanoma which does not respond to a treatment. The cancer may be resistant at the beginning of treatment, or it may become resistant during treatment. The resistance to drug leads to rapid progression of metastatic of melanoma.

As used herein, the term “resistant melanoma cell” refers to cell which does not respond to a treatment. As used herein, the term “sensitive melanoma cell” refers to cell which does respond to a treatment.

In some embodiments, the melanoma is resistant to BRAF inhibitors. BRAF is a member of the Raf kinase family of serine/threonine-specific protein kinases. This protein plays a role in regulating the MAP kinase/ERKs signaling pathway, which affects cell division, differentiation, and secretion. A number of mutations in BRAF are known. In particular, the V600E mutation is prominent. Other mutations which have been found are R461I, 1462S, G463E, G463V, G465A, G465E, G465V, G468A, G468E, N580S, E585K, D593V, F594L, G595R, L596V, T598I, V599D, V599E, V599K, V599R, K600E, A727V, and most of these mutations are clustered to two regions: the glycine-rich P loop of the N lobe and the activation segment and flanking regions. In a particular embodiment, the BRAF mutation is V600E.

The inhibitors of BRAF mutations are well known in the art.

In some embodiments, the melanoma is resistant to MEK inhibitors. MEK refers to Mitogen-activated protein kinase kinase, also known as MAP2K, MEK, MAPKK. It is a kinase enzyme which phosphorylates mitogen-activated protein kinase (MAPK). MEK is activated in melanoma.

In some embodiments, the melanoma is resistant to NRAS inhibitors. The NRAS gene is in the Ras family of oncogene and involved in regulating cell division. NRAS mutations in codons 12, 13, and 61 arise in 15-20% of all melanomas.

In some embodiments, the melanoma is resistant to immune checkpoint inhibitors.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCNI, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tcl function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolismoLiu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo (b) thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo (b) thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophényl)-N′-hydroxy-4-{[2-(sulfamoylamino)-ethyl]amino}-1,2,5-oxadiazole-3 carboximidamide:

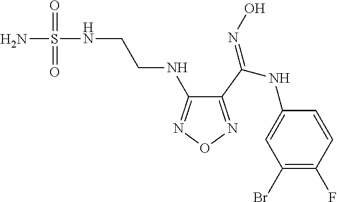 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7] cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

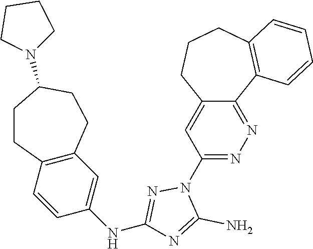 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

In a particular embodiment, the melanoma is metastatic uveal melanoma.

As used herein, the term “metastasis” refers to the spread of cancer cells from a primary site and the formation of new tumors in another region of the body. Metastasis is responsible for as much as 90% of cancer-associated mortality. The liver is often the first metastatic site in patients with uveal melanoma. Accordingly, metastatic uveal melanoma refers migration of ciliary or choroid cells to the liver and induces liver metastasis.

In a particular embodiment, the resistant melanoma is uveal resistant melanoma.

As used herein, the term “uveal melanoma resistant” refers to uveal melanoma which does not respond to a treatment. The cancer may be resistant at the beginning of treatment or it may become resistant during treatment. The resistance to drug leads to rapid progression of metastatic of uveal melanoma.

The resistance of cancer for the medication is caused by mutations in the gene which are involved in the proliferation, divisions or differentiation of cells.

In a particular embodiment, the uveal melanoma resistant has at least one mutation in the five following genes: BAP1, EIF1AX, GNA11, GNAQ, and/or SF3B1.

In a particular embodiment, the resistant melanoma is resistant to to a treatment with an immune check point inhibitor as described above.

### Combined Preparation

In a third aspect, the invention relates to i) an iron metabolism disruptor and ii) a classical treatment as a combined preparation for use in the treatment of melanoma and/or resistant melanoma.

In a particular embodiment, the iron metabolism disruptor is salinomycin and its derivatives.

In a particular embodiment, the iron metabolism disruptor is ironomycin.

In a further embodiment, the invention relates to i) salinomycin and its derivatives and ii) a classical treatment as a combined preparation for use in the treatment of melanoma and/or resistant melanoma.

In a particular embodiment, the invention relates to i) salinomycin and its derivatives and ii) a classical treatment as a combined preparation for use in the treatment of uveal melanoma.

In a particular embodiment, the invention relates to i) salinomycin and its derivatives and ii) a classical treatment as a combined preparation for use in the treatment of uveal resistant melanoma.

In a particular embodiment, the invention i) salinomycin and its derivatives and ii) a classical treatment as a combined preparation for simultaneous, separate or sequential use in the treatment of melanoma.

In a particular embodiment, the invention i) salinomycin and its derivatives and ii) a classical treatment as a combined preparation for simultaneous, separate or sequential use in the treatment of uveal melanoma and/or uveal resistant melanoma.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

As used herein, the term “classical treatment” refers to treatments well known in the art and used to treat melanoma. In the context of the invention, the classical treatment refers to targeted therapy, radiation therapy, chemotherapy immunotherapy, HDAC inhibitor or calcium channel blocker CCB.

As used herein, the term “targeted therapy” refers to drugs which attack specific genetic mutations within cancer cells, such as melanoma while minimising harm to healthy cells. Typically, the targeted therapy for melanoma refers to use of BRAF, MEK or NRAS inhibitors as described above.

As used herein, the term “immunotherapy” has its general meaning in the art and refers to the treatment that consists in administering an immunogenic agent i.e. an agent capable of inducing, enhancing, suppressing or otherwise modifying an immune response. In a particular embodiment, the immunotherapy consists of use of an immune check point inhibitor as described above.

As used herein, the term “chemotherapy” refers to use of chemotherapeutic agents to treat a subject. As used herein, the term “chemotherapeutic agent” refers to chemical compounds that are effective in inhibiting tumor growth.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In a particular embodiment, the invention relates to i) salinomycin and its derivatives and ii) an histone deacetylase inhibitor as a combined preparation for simultaneous, separate or sequential use in the treatment of uveal melanoma and/or uveal resistant melanoma.

As used herein, the term histone “histone deacetylase inhibitor” called also HDACi, refers to a class of compounds that interfere with the function of histone deacetylase. Histone deacetylases (HDACs) play important roles in transcriptional regulation and pathogenesis of cancer. Typically, inhibitors of HDACs modulate transcription and induce cell growth arrest, differentiation and apoptosis. HDACis also enhance the cytotoxic effects of therapeutic agents used in cancer treatment, including radiation and chemotherapeutic drugs. In a particular embodiment, the histone deacetylase inhibitor is valproic acid (VPA). The term “valproic acid” refers to acid-2-propylpentanoic (C₈H₁₆O₂), 5 which has the following CAS number and formula 99-66-1 in the art:

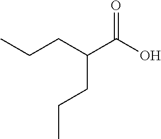

In a particular embodiment, the HDAC inhibitor is suberoylanilide hydroxamic acid, also called Vorinostat (N-Hydroxy-N′-phenyloctanediamide) was the first histone deacetylase inhibitor approved by the U.S. Food and Drug Administration (FDA) on 2006 (Marchion D C et al 2004; Valente et al 2014).

In a particular embodiment the HDAC inhibitor is Panobinostat (LBH-589) has received the FDA approval on 2015 and has the structure as described in Valente et al 2014.

In a particular embodiment the HDAC inhibitor is Givinostat (ITF2357) has been granted as an orphan drug in the European Union (Leoni et al 2005; Valente et al 2014).

In a particular embodiment the HDAC inhibitor is Belinostat also called Beleodaq (PXD-101) has received the FDA approval on 2014 (Ja et al 2003; Valente et al 2014).

In a particular embodiment the HDAC inhibitor is Entinostat (as SNDX-275 or MS-275). This molecule has the following chemical formula (C₂₁H₂₀N₄O₃) and has structure as described in Valente et al 2014.

In a particular embodiment the HDAC inhibitor is Mocetinostat (MGCD01030) having the following chemical formula (C₂₃H₂₀N₆O) (Valente et al 2014).

In a particular embodiment the HDAC inhibitor is Practinostat (SB939) having the following chemical formula (C₂₀H₃₀N₄O₂) and the structure as described in Diermayr et al 2012. In a particular embodiment the HDAC inhibitor is Chidamide (CS055/HBI-8000) having the following chemical formula (C₂₂H₁₉FN₄O₂).

In a particular embodiment the HDAC inhibitor is Quisinostat (JNJ-26481585) having the following chemical formula (C₂₁H₂₆N₆O₂).

In a particular embodiment the HDAC inhibitor is Abexinostat (PCI24781) having the following chemical formula (C₂₁H₂₃N₃O₅) (Valente et al 2014).

In a particular embodiment the HDAC inhibitor is CHR-3996 having the following chemical formula (C₂₀H₁₉FN₆O₂) (Moffat D et al 2010; Banerji et al 2012).

In a particular embodiment the HDAC inhibitor is AR-42 having the following chemical formula (C₁₈H₂₀N₂O₃) (Lin et al 2012).

In a particular embodiment, the invention relates to i) salinomycin and its derivatives and ii) a calcium channel blocker as a combined preparation for simultaneous, separate or sequential use in the treatment of uveal melanoma and/or uveal resistant melanoma.

As used herein, the term “calcium channel blocker” (CCB) refers to calcium channel antagonists or calcium antagonists that disrupt the movement of calcium (Ca2+) through calcium channels.

In a particular embodiment, the calcium channel blocker is selected from the following group consisting of but not limited to Amlodipine (Norvasc), Aranidipine (Sapresta), Azelnidipine (Calblock), Barnidipine (HypoCa), Benidipine (Coniel), Cilnidipine (Atelec, Cinalong, Siscard), Clevidipine (Cleviprex), Efonidipine (Landel), Felodipine (Plendil), Isradipine (DynaCirc, Prescal), Lacidipine (Motens, Lacipil), Lercanidipine (Zanidip), Manidipine (Calslot, Madipine), Nicardipine (Cardene, Carden SR), Nifedipine (Procardia, Adalat), Nilvadipine (Nivadil), Nimodipine (Nimotop), Nisoldipine (Baymycard, Sular, Syscor), Nitrendipine (Cardif, Nitrepin, Baylotensin), Pranidipine (Acalas), Fendiline, Gallopamil, Verapamil (Calan, Isoptin) or Diltiazem.

### Pharmaceutical Composition

The iron metabolism disruptor for use according to the invention alone and/or combined with classical treatment as described above may be combined with pharmaceutically acceptable excipients, and optionally sustained-release matrices, such as biodegradable polymers, to form pharmaceutical compositions.

Accordingly, in a fourth aspect, the invention relates to a pharmaceutical composition comprising an iron metabolism disruptor for use in the treatment of melanoma.

In a particular embodiment, the pharmaceutical composition according to the invention for use in the treatment of uveal melanoma and/or uveal resistant melanoma.

In a particular embodiment, the pharmaceutical composition according to the invention comprising salinomycin and its derivatives for use in the treatment of melanoma and/or resistant melanoma.

In a particular embodiment, the pharmaceutical composition according to the invention comprising salinomycin and its derivatives for use in the treatment of uveal melanoma and/or uveal resistant melanoma.

In a particular embodiment, the pharmaceutical composition according to the invention comprising i) an iron metabolism disruptor and ii) a classical treatment, as a combined preparation for use in the treatment of melanoma and/or resistant melanoma.

In a particular embodiment, the pharmaceutical composition according to the invention comprising i) salinomycin and its derivatives and ii) a classical treatment, as a combined preparation for use in the treatment of melanoma and/or resistant melanoma.

In a particular embodiment, the pharmaceutical composition according to the invention comprising i) iron metabolism disruptor and ii) a classical treatment, as a combined preparation for use in the treatment of uveal melanoma and/or uveal resistant melanoma.

In a particular embodiment, the pharmaceutical composition according to the invention comprising i) salinomycin and its derivatives and ii) a classical treatment, as a combined preparation for use in the treatment of uveal melanoma/and or uveal resistant melanoma.

The iron metabolism disruptor and the combined preparation as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intravitreal administration, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

### Method of Screening

In another aspect, the present invention relates to a method of screening a drug suitable for the treatment of melanoma, aggressive/invasive melanoma, metastatic melanoma, or melanoma resistant, uveal melanoma or uveal resistant melanoma comprising i) providing a test compound and ii) determining the ability of said test compound to inhibit the expression and/or activity of iron metabolism.

Typically, such test compound is able to inhibit the expression and/or activity of inhibitor of iron metabolism.

Any biological assay well known in the art could be suitable for determining the ability of the test compound to inhibit iron metabolism. In some embodiments, the assay first comprises determining the ability of the test compound to bind to iron metabolism. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of iron metabolism, as determined using in vitro methods described herein, are likely to exhibit similar modulatory capacity in applications in vivo. Typically, the test compound is selected from the group consisting of peptides, petptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: Ironomycin resensitizes PLX-resistant metastatic cutaneous melanoma cells to PLX effects. PLX-sensitve and resistant cutaneous melanoma cells, seeded at 150,000 cells/well in 6-well dishes, were treated with increasing concentrations of AM23 or AM5 in presence or absence of PLX (5 mM). Cells were manually counted after 72 h.

FIG. 2: Salinomycin or AM23 induces uveal melanoma cell death. Uveal melanoma cells (OMM1.3, MM28, MP46, MP65) were seeded at 5,000 cells/well in a 96-well plate and treated with increasing concentrations of AM23, salinomycin or staurosporine as positive control as indicated on the figure. The condition “milieu RPMI” represents medium without cells. After 96 h of incubation, cells were incubated with PrestoBlue reagent according to manufacture instructions. The absorbance per well was measured with a microplate reader. The data were then analyzed using excel.

FIG. 3: AM23 prevents melanosphere formation. Uveal melanoma cells (OMM1.3, MM28, MP65 and MP46) were seeded at 5000 cells/well in 6-well low attachment plates and were left untreated (0) or were treated with DMSO (Ctl) or AM23 (100 nM or 1 mM) 24 hours later. Representative pictures taken after 8 days are shown.

FIG. 4: AM23 and salinomycin induces Fe (ii) in lysosomes. A. MP46 and B. OMM1.3 human uveal melanoma cells were seeded at 5000 cells/well on a glass coverslip and were treated with DMSO (Ctl), AM23 (30 nM or 100 nM) or salinomycin (0.5 mM) for 48 h and then with RhoNox-M for 1 h for Fe 2+ detection. Representative confocal fluorescence microscopy images.

FIG. 5: Effect of AM23 or salinomycin on ferritin expression. OMM1.3 and MP46 human uveal melanoma cells were left untreated (UNT) or were treated with DMSO (Ctl), AM23 (100 nM) or salinomycin (0.5 mM) for 48 h. Cell lysates were analysed by immunoblot with antibodies to ferritin. Actin was used as a loading control.

FIG. 6: Colony formation assay of primary uveal melanoma cells (MP46 and MP65) or metastatic uveal melanoma cells (OMM1.3) expressing or not BAP1 after seeding them for 7 days upon treatment with the drugs indicated on the figure. Representative wells are shown.

FIG. 7: Primary (MP46) or metastatic (OMM1, OMM2.5, OMM1.3) uveal melanoma cells were seeded at 3000 cells/well in a 96 well plate and treated with increasing concentrations of salinomycin, AM23 or AM5. After 72 h of incubation, cells were treated with PrestoBlue reagent according to manufacture instructions. The absorbance per well was measured with a microplate reader. The data were then analyzed using Prism8. Histograms bars represent the average of three replicates #standard error of the mean (s.e.m).

### EXAMPLE

### Material & Methods

### Cell Count

PLX-sensitve and resistant cutaneous melanoma cells were seeded at 150,000 cells/well in 6-well dishes. They were treated with increasing concentrations of AM23 or AM5 in presence or absence of PLX (5 M). Cells were manually counted after 72 h.

### Cell Survival Assay

Uveal melanoma cells were seeded at 5,000 cells/well in a 96-well plate and treated with salinomycin or ironomycin as well as a positive control (staurosporin, 500 nM). After 72 h of incubation, cells were treated with PrestoBlue reagent according to manufacture instructions (Thermofisher). The absorbance per well was measured with a microplate reader. The data were then analyzed using excel software.

### Spheroid Formation

Uveal melanoma cells were seeded at a density of 5,000 cells per well and grown in MEF medium (R&D systems, #AR005) supplemented with 4 ng/ml FGF2 in low adherence plates. The following day, wells were supplemented with ironomycin (100 nM or 1 mM). Photographs were taken using a phase contrast microscope (EVOS XL Core Imaging System).

### Western Blot Assays

Briefly, uveal melanoma cell lysates (50 μg) were separated using SDS-PAGE, transferred onto a PVDF membrane and subsequently exposed to the appropriate antibodies, anti-ferritin (#sc376594, 1/1,000) and anti-actin (sc47778, 1/1,000) from Santa Cruz Biotechnology. The proteins were visualized using the ECL system (Amersham). The western blots shown are representative of at least 3 independent experiments.

### Results

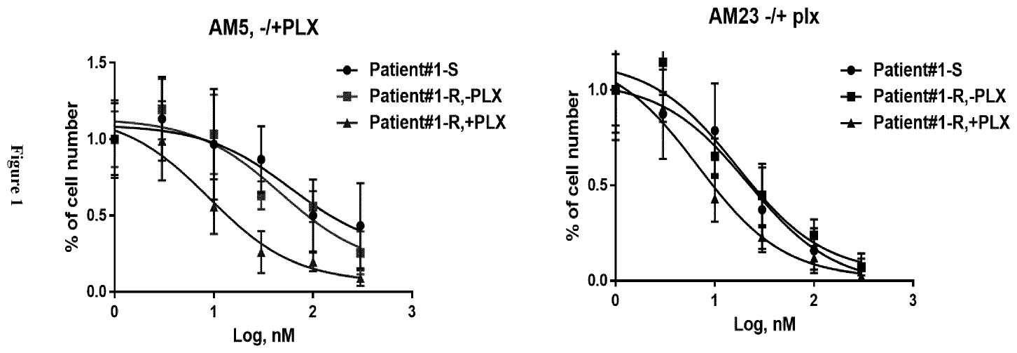

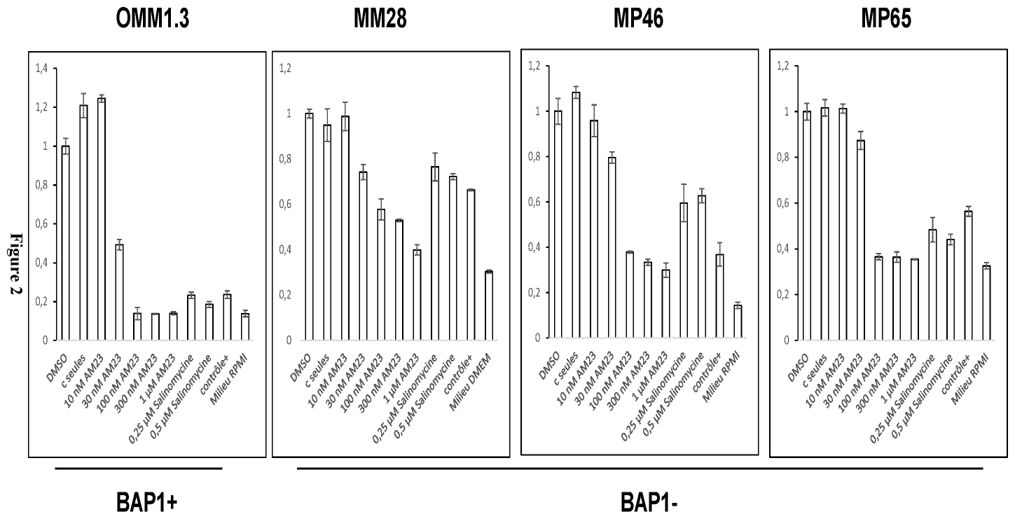

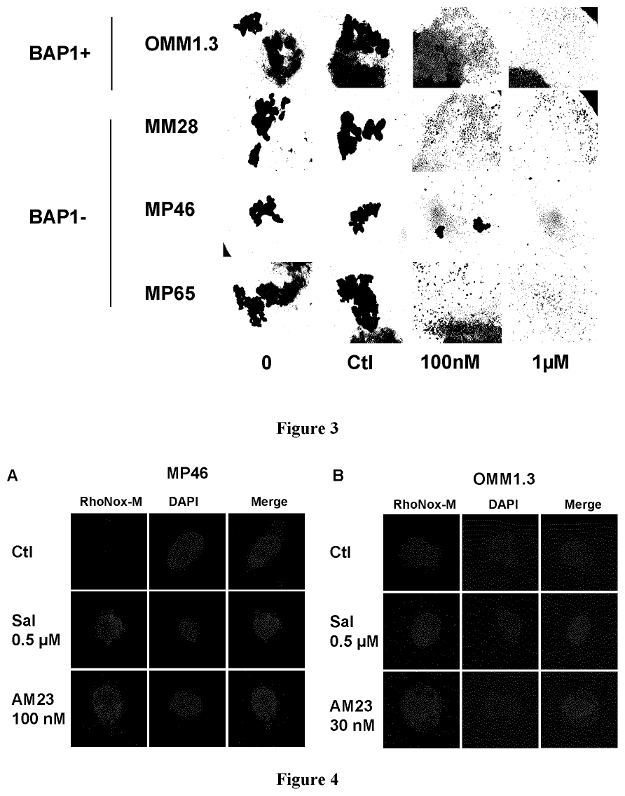

Uveal melanoma cells are known to hardly enter into apoptosis in response to treatments. Thus, we investigated the effects of salinomycin (Sal) and its derivate called ironomycin (AM23) that have been shown, in different cancer cells, to trigger ferroptosis, a type of cell death based on accumulation of iron into lysosomes and subsequent lysosomal membrane permeabilization. We treated a panel of uveal melanoma cells, i.e the human metastatic OMM1.3 (BAP1+) and MM28 (BAP1+) cells, and the human primary MP46 (BAP1−) and MP65 (BAP1−) cells, with Sal or its derivate AM23 to determine if these drugs can represent potential therapeutic options. Data show that both Sal and AM23 reduced proliferation/survival of uveal melanoma cell in 2D-cultures (FIG. 1). Additionally, as observed by almost complete disappearance of melanospheres at 100 nM, AM23 was as well highly effective in reducing uveal melanoma cell survival in 3D-cultures that much more closely resemble the cells growing within living tumors (FIGS. 2 and 3).

To assess whether AM23 triggers lysosomal iron accumulation, we evaluated whether endocytic vesicles contained Fe (ii) as previously reported. To this aim, the Fe (ii)-specific turn-on fluorescent probe RhoNox-M, which can detect the presence of Fe (ii) in lysosomes, was used. As shown by microscopy, uveal melanoma cells exposed to Sal or AM23 exhibited fluorescent RhoNox-M-positive vesicles compared to control cells in which fluorescence was weak and diffuse (FIG. 4). These data indicate that Sal or its derivate AM23 causes iron accumulation in lysosomes. In response to the ensuing cytoplasmic depletion of iron, cells triggered in lysosomes the degradation of ferritin, which stores cellular iron, leading to further iron loading in this organelle.

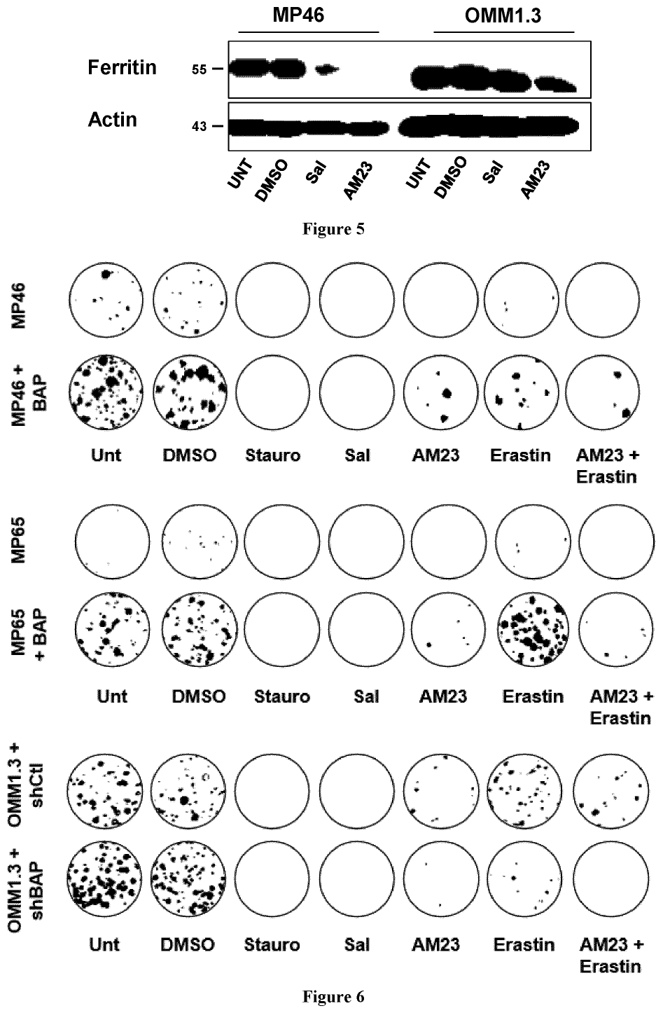

Supporting the idea that ferroptosis is at play in uveal melanoma cells, ferritin expression was strongly reduced in presence of both Sal and AM23 (FIG. 5).

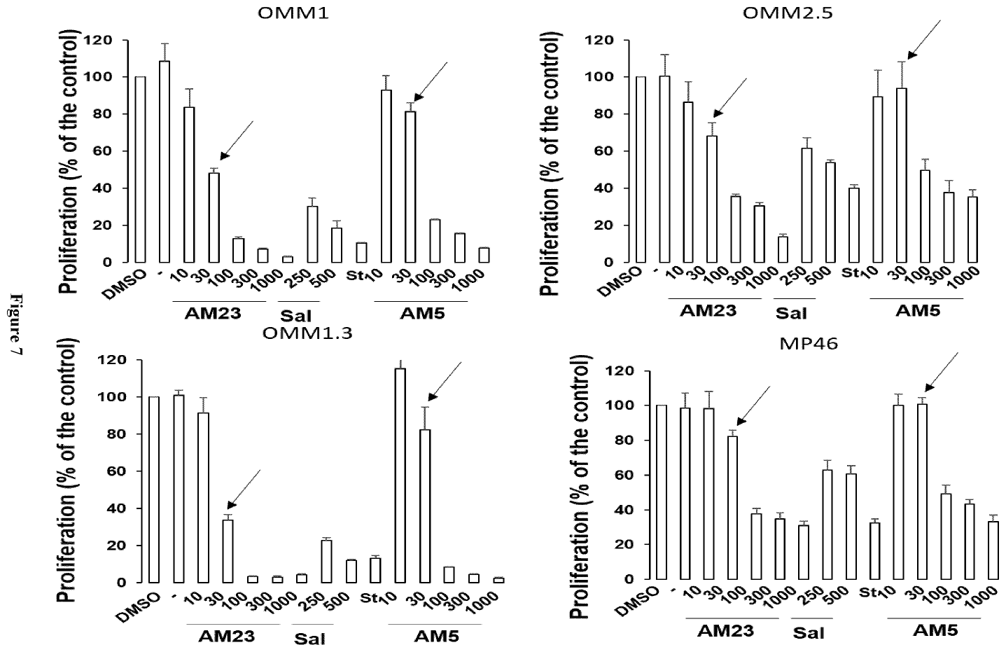

The results also show that Sal and AM23 induces a strong reduction in the ability of uveal melanoma cells to form colonies whether BAP1 is expressed or not (FIG. 6). Finally, we tested the effect of another Sal derivatives, AM5. AM5 also decreases uveal melanoma cell proliferation/survival, yet with a little bit lower efficacy than AM23 (FIG. 7).

Collectively, our results suggest that Sal and AM23, through lysosomal iron accumulation, reduced proliferation/survival of uveal melanoma cells harboring different genetic background. In conclusion, salinomycin and its derivates ironomycin represent valuable therapeutic strategies to treat uveal melanoma cells.

### REFERENCES

Throughout this application, various references describe the state of the art to which this invention pertains. The disclosures of these references are hereby incorporated by reference into the present disclosure.

1. A method for treating melanoma in a subject in need thereof comprising administering to said subject a therapeutically effective amount of an iron metabolism disruptor.

2. The method according to claim 1 wherein the melanoma is uveal melanoma.

3. The method according to claim 1 wherein the melanoma is uveal melanoma resistant.

4. The method according to claim 1 wherein the iron metabolism disruptor is salinomycin or a derivative thereof.

5. The method according to claim 4 wherein the derivate of salinomycin is AM23.

6. The method according to claim 1 wherein the iron metabolism disruptor is ironomycin.

7. The method according to claim 1 wherein the iron metabolism disruptor is administered by topical or intravitreal administration.

8. The method of claim 1, wherein the iron metabolism disruptor is administered with a classical melanoma treatment agent in a combined preparation.

9. The method according to claim 8 wherein the melanoma is uveal melanoma.

10. The method according to claim 8 wherein the iron metabolism disruptor is salinomycin or a derivative thereof.

11. A pharmaceutical composition comprising an iron metabolism disruptor formulated for use in the treatment of melanoma and/or uveal melanoma.

12. The pharmaceutical composition according to claim 11 further comprising a classical melanoma treatment agent.

13. The pharmaceutical composition according to claim 11, wherein the iron metabolism disruptor is salinomycin or a derivative thereof.

14. A method of screening a drug suitable for the treatment of melanoma comprising i) providing a test compound ii) determining the ability of said test compound to inhibit the expression and/or the activity of iron metabolism and iii) selecting the test compound as suitable for the treatment of melanoma when the test compound inhibits the expression and/or the activity of iron metabolism.
